(19) United States
(12) Patent Application Publication
(10) Pub. No.: US 2011/0066172 A1
(43) Pub. Date: Mar. 17, 2011

(54) EAR CLEANING SYSTEM AND METHOD

(76) Inventor: Herbert Silverstein, Sarasota, FL (US)

(21) Appl. No.: 12/879,095

(22) Filed: Sep. 10, 2010

Related U.S. Application Data
(60) Provisional application No. 61/241,978, filed on Sep. 14, 2009.

(51) Int. Cl. A61F 11/00

(52) U.S. Cl. 606/162

(57) ABSTRACT

A device for cleaning the ear canal of cerumen or contaminants includes a handle, a material expandable upon exposure to a fluid, and a limiter connected to the handle operative to prevent insertion of the expandable material into the ear to a depth which could contact the ear drum. Fluid is passed through the handle and through apertures to the ear canal, and or to the expandable material, whereby the ear canal and expandable material are moistened. The expandable material expands to contact the ear canal walls and cerumen, whereby the cerumen may be extracted by withdrawing the handle and connected expandable material from the ear. The fluid may be an alcohol solution, which loosens cerumen, reduces the potential for infection, and promotes drying of the ear canal.

### CROSS REFERENCE TO RELATED APPLICATIONS

This application claims the benefit of U.S. Provisional Application 61/241,978 to the same inventor, filed Sep. 14, 2009, entitled “Ear Canal Cleaning System and Method”, the entire contents of which are incorporated herein by reference.

### FIELD OF THE INVENTION

The invention relates to cleaning of ears, and more particularly the removal of contaminants and cerumen from the ear canal using a liquid and mechanical means.

### BACKGROUND OF THE INVENTION

Normally, the ear canal in humans and many mammals produces a substance called cerumen, and commonly termed “ear wax”. The cerumen may take a predominantly dry or wet form, which has been found to be related to genetics, to an extent. Glands that produce cerumen are located in the outer portion of the ear canal. An amount of cerumen may be removed from the outer portion of the ear canal, in some cases, by wiping a damp cloth in the outer portion of the ear.

The ear has a natural ability to clean itself, which causes the migration of excess cerumen towards the end of the ear canal. This natural ability can be impaired in smaller ear canals, or by the presence of mechanical hearing aids, or by inserting other objects into the ear. An accumulation of cerumen may cause discomfort, and can eventually lead to a reduced ability to hear.

Additionally, the insertion of elongated objects, such as cotton swabs, endanger the eardrum, raise the potential for infection, and tend to push accumulated cerumen deeper into the ear canal, resulting in hearing impairment.

One method of cerumen removal is the use of a standard bulb syringe to direct water into the ear canal. This is often done by a medical professional, particularly when the buildup of cerumen has caused difficulties.

The art described in this section is not intended to constitute an admission that any patent, publication or other information referred to herein is “prior art” with respect to this invention, unless specifically designated as such. In addition, this section should not be construed to mean that a search has been made or that no other pertinent information as defined in 37 CFR §1.56(a) exists.

### SUMMARY OF THE INVENTION

The invention enables expeditious cleaning of cerumen and or contaminants from the ear and ear canal, without causing injury to ear tissue. In accordance with the invention, a contact member including an expandable material is positioned at a distal end of a device, which swells when exposed to a fluid. In a non-expanded state, the expandable material readily inserts into the ear canal. When swollen or expanded, at least portions of the expandable material contact the wall of the ear canal such that motion, such as rotation or twisting, cleans the ear canal wall. The expandable material can be any material that swells when exposed to an irrigation or therapeutic fluid.

A device in accordance with the invention includes a handle adapted to be grasped by the hand of a user. A fluid reservoir and passage is disposed within the handle, operative to contain or conduct fluid. A limiter is connected to an end of the handle, operative to contact the tragus of the ear, thereby preventing insertion of the device too deeply into the ear canal. A support tube extends from the limiter, and is operative to conduct fluid delivered from the passage, through the limiter. An expandable ear canal contacting member is connected to the tube, and includes an expandable material operative to increase in size when exposed to a fluid.

In use, the device of the invention is inserted into the ear canal, until a leading face of limiter contacts the tragus, or cartilaginous structure protecting the opening of the ear canal. Once the device is inserted into the ear canal, fluid is passed from the passage, and through or around the limiter, whereupon the fluid exits through one or more apertures disposed in tube. Apertures are oriented to conduct fluid to contact side walls of the ear canal. Accordingly, fluid flow does not directly impact the ear drum, for safety and comfort.

The dimensions of the device, and particularly the tube length extending from the limiter, and the size of the expandable material, are advantageously selected to fit a particular ear size, for example a child or an adult of the species being treated.

Expandable material of the contact member absorbs fluid expelled through the apertures, and expands to contact side wall portions of the ear canal. In so doing, the expandable material contacts a cerumen accumulation, and mechanically transfers the cerumen to the expandable material with movement of the device.

A fluid reservoir may be connected to the device, operative to pass a cleaning or therapeutic fluid to the passage, and ultimately to the apertures and the expandable material. If the reservoir is provided in the form of a dropper bottle, a mating tapered connection at opening may be advantageous. Alternatively, the passage may be sized and shaped to be filled with fluid prior to being provided to the user, and passage may be sealed at a point of manufacture.

In another embodiment of the invention, an ampule or other fluid chamber may be positioned within the passage. In this event, the inner chamber is squeezed within the passage and thus broken, releasing a contained fluid within passage. This is advantageous in the event that materials must be mixed just prior to use. In this embodiment, one or more materials to be mixed are placed within the passage, for example a powder or a different liquid, and the remaining elements of a mixture are contained within chamber.

Expandable material may be formed into a square or rectangular shape, or any other desired shape. It is advantageous, however, to round or soften any sharp edges, for the comfort and safety of the user. In one alternative embodiment of the invention, at least a portion of an end of tube is opened, or apertures are provided along a portion of the tube, within the expandable material, so that fluid may flow into expandable material.

In yet another embodiment of the invention, expandable material has the form of a brush of bristles. When not moistened or wet, the brush is sufficiently narrow to be inserted into the ear canal without driving contaminants further into the ear canal. When the bristles have been moisted, the expandable material of the bristles expand to improves a contact of the expandable material with inner sides of the ear canal, and any contaminants or cerumen accumulated within ear canal.

The handle, limiter, tube, and other components of a device in accordance with the invention may be formed or molded integrally, or may be joined by any known means, including heat, adhesive, ultrasonic energy, or fasteners. Biocompatible and or biodegradable materials are advantageous used for health and ecological benefits. Accordingly, polymeric materials are advantageous, and may be used to form a device which is economically disposable. Alternatively, devices in accordance with the invention may be formed with metal, glass, or other durable material, whereby the device may be cleaned or sterilized by any known means, and the contacting member may be advantageously replaced for each patient.

In one embodiment of the invention, the expandable material is a compressed or uncompressed natural or synthetic material, advantageously provided in a sponge structure, operative to expand when contacted by a fluid, for example water; an alcohol solution; a sodium bicarbonate solution; an antibiotic, antiseptic, or anesthetic solution; or other therapeutic liquid substance. The material is advantageously selected for a rate, force and extent of expansion which is comfortable and safe for the ear, the selection of which, beyond the examples suggested herein, is within the abilities of one skilled in the art.

A variety of substances, in accordance with the invention, may be advantageously used to facilitate removal of cerumen and to clean the ear canal. In this regard, 6.5% carbamide peroxide in an anhydrous glycerin base can be used, and is particularly effective for cerumen removal. In one embodiment, a solution containing 70% isopropyl alcohol is used to soften cerumen, as well as to reduce a potential for infection within the ear, and to promote a reduction of moisture within the ear. In another embodiment, 95% isopropyl alcohol is used, for example in an anhydrous glycerin 5% base. A range of alcohol concentrations may advantageously be used for these purposes, including less than 1% to more than 99% alcohol, depending upon the patient's condition and comfort level, and the therapeutic goals of the practitioner.

Alcohol may also be combined with boric acid, for example about 3% boric acid in about 70% alcohol, to treat most bacterial and fungal infections. This solution, or any other solution introduced by the device of the invention into ear canal, may be maintained in place, if desired, by placing ear putty at the opening of ear canal.

In one embodiment of the invention, sodium bicarbonate may be placed in an attached fluid chamber, whereby breakage or opening of a fluid chamber internally disposed within the device exposes the sodium bicarbonate to a fluid, for example water within, or introduced into the passage, to hydrate and activate the material, so that it may convey a therapeutic benefit, including for example a reduction in pH, a reduction of an infection or allergic reaction, or for cleaning. Alternatively, a liquid may be contained within the fluid chamber, and sodium bicarbonate may be placed within the passage. In a similar manner, other chemicals may be combined with liquids and or other chemicals, to produce a fresh composition which offers a therapeutic benefit when introduced into the ear canal.

The fluid reservoir or tube is squeezed to pass fluid quickly and with sufficient pressure to loosen cerumen and moisten expandable material, but not with such excess pressure that might cause discomfort, or damage to the ear. The cleaning process is repeated until, if desired, the effluent is free of cerumen or contaminants. Optionally, cleaning may be conducted with sufficient frequency, for example weekly, to maintain the ear canal clean, dry, and free of infections.

In yet another embodiment of the invention, apertures may be sized sufficiently small to produce a mist when a liquid is driven therethrough at sufficient pressure. Additionally, a sufficient quantity of apertures may be provided to produce a sufficient quantity of mist over a desired area, for example, to mist the a substantial portion of the ear canal.

Accordingly, a device of the invention for cleaning the ear canal, comprises: a handle having a distal and a proximal end; a contact member including a material expandable from a first smaller size to a second larger size upon contact with a fluid, the contact member connected to the proximal end of the handle and sized to be smaller than the ear canal when the expandable material has the first smaller size, whereby the expandable material is insertable into the ear canal; a limiter connected to the handle, disposed between the handle distal end and the contact member, the limiter including a face having a diameter greater than an opening to the ear canal, whereby the limiter is operative to limit a depth of insertion of the handle and the contact member; means for passing a fluid through the handle to the contact member when the contact member is disposed within the ear canal, whereby the contact member may be thereby expanded from the first smaller size to the second larger size, whereupon the contact member is sized to contact a wall of the ear canal; wherein the handle is movable together with the connected contact member to thereby dislodge cerumen or contaminants from attachment to a wall of the ear canal.

Alternatives of the device in accordance with the invention further include one or more embodiments wherein: a support extension disposed between the limiter and the contact member is operable to support the contact member within the ear canal, and to conduct a fluid; one or more apertures disposed between the limiter and the contact member, operative to direct fluid to a wall of the ear canal and to the contact member; apertures direct fluid to a wall of the ear canal at an angle less than 90 degrees with respect to an angle of a wall of the ear canal; apertures direct fluid into an interior portion of the expandable material; the handle includes a fluid passage operative to contain a fluid and pass the fluid from a distal portion of the handle to a proximal portion of the handle; the handle includes a fluid passage operative to contain a fluid and pass the fluid, from a distal portion of the handle to the ear canal, under pressure; the second larger size of the contact member has a diameter greater than a diameter of the ear canal; the handle and the contact member are not provided with a means for mutual relative disengagement; the expandable material is selected from the group consisting of: sponge, cellulosic material, polyvinyl acetate, Merocel, polymer, natural fiber, synthetic fiber; the expandable material has a textured or roughened exterior surface; a reservoir is disposable within the handle, the reservoir operable to be ruptured to release a first material into an interior in the handle; the interior in the handle is configured to contain a second material, and wherein a combination of the first material and the second material produces a therapeutic substance; a reservoir is connectable to the handle, operative to introduce a material into the fluid passage when the reservoir is connected to the handle; the expandable material has the form of a brush; the handle and the limiter are not provided with a means for mutual releasable engagement; the expandable material is compressed when having the first smaller size; and, the fluid is selected from the group consisting of: alcohol, isopropyl alcohol solution, an isopropyl alcohol and water solution, water, sodium bicarbonate solution, an antibiotic in solution, an antiseptic in solution.

In a further embodiment of the invention, a device for cleaning the ear canal, comprises: a handle having a distal and a proximal end; a contact member including a material expandable from a first smaller size to a second larger size upon contact with a fluid, the contact member connected to the proximal end of the handle and sized to be smaller than the ear canal when the expandable material has the first smaller size, whereby the expandable material is insertable into the ear canal; a limiter connected to the handle, disposed between the handle distal end and the contact member, the limiter including a face having a diameter greater than an opening to the ear canal, whereby the limiter is operative to limit a depth of insertion of the handle and the contact member; a support extension extending between the limiter and the contact member, provided with apertures operative to direct a fluid in a direction of the ear canal when at least a portion of the support extension is disposed within the ear canal, whereby the contact member may expanded, by contact with a fluid, from the first smaller size to the second larger size, whereupon the contact member is sized to contact opposing walls of the ear canal; wherein the handle is movable together with the connected contact member to thereby dislodge cerumen or contaminants from attachment to a wall of the ear canal.

In yet another embodiment of the invention, a device for cleaning the ear canal, comprises a handle having a distal and a proximal end; a contact member including a material expandable from a first smaller size to a second larger size upon contact with a fluid, the contact member connected to the proximal end of the handle and sized to be smaller than the ear canal when the expandable material has the first smaller size, whereby the expandable material is insertable into the ear canal; a limiter connected to the handle, disposed between the handle distal end and the contact member, the limiter including a face having a diameter greater than an opening to the ear canal, whereby the limiter is operative to limit a depth of insertion of the handle and the contact member; a support extension extending between the limiter and the contact member, provided with at least one aperture operative to direct a fluid in a direction of the ear canal when at least a portion of the support extension is disposed within the ear canal, whereby the contact member may expanded, by contact with a fluid, from the first smaller size to the second larger size, whereupon the contact member is sized to contact opposing walls of the ear canal; a reservoir connectable with the handle, operative to contain a fluid and pass the fluid to the at least one aperture; wherein the handle is movable together with the connected contact member to thereby dislodge cerumen or contaminants from attachment to a wall of the ear canal.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, in which:

FIG. 1 illustrates an ear cleaning device in accordance with the invention;

FIG. 2 illustrates the ear cleaning device of FIG. 1, after exposure to a liquid;

FIG. 3 illustrates the ear cleaning device of FIG. 1, positioned proximate an ear;

FIG. 4 illustrates the ear cleaning device of FIG. 1, a portion of which inserted into an ear canal;

FIG. 5 illustrates an enlarged view of the ear cleaning device of FIG. 1, further illustrating fluid passages within an expandable ear canal contacting member of the invention;

FIG. 6 illustrates the contacting member of FIG. 5, expanded within the ear;

FIG. 7 illustrates an embodiment of the invention in which a fluid is introduced into a device of the invention, through an aperture at an end of the device, through a connection with a fluid reservoir;

FIG. 7A illustrates a fluid reservoir and internal tube in accordance with the invention;

FIG. 8 illustrates a fluid container within a device in accordance with the invention, and a sealing member;

FIG. 9 illustrates a perspective view of a device in accordance with the invention, including an ear contacting member in the form of a pad;

FIG. 10 illustrates a reverse view of the device of FIG. 9;

FIG. 11 illustrates a contacting member stiffening device, in the form of a flexible pin within the contacting member;

FIG. 12A illustrates a contacting member having the form of a brush;

FIG. 12B illustrates the brush of FIG. 12A, expanded by exposure to a liquid;

FIG. 13 illustrates the brush of FIG. 2 with an aperture for release of fluids only within an expandable material; and

FIG. 14 illustrates the brush of FIG. 2, with a single aperture at a tube end within the expandable material.

All dimensions shown in the figures or discussed in this specification are illustrative only, are not to scale, and should not be considered limiting, as the present invention contemplates various configurations.

### DETAILED DESCRIPTION OF THE INVENTION

The invention enables expeditious cleaning of cerumen and or contaminants from the ear and ear canal 44, without causing injury to ear tissue, including the ear canal 44 and ear drum 48. In accordance with the invention, a contact member 20 including an expandable material 22 is positioned at a distal end of device 10, and swells when exposed to a fluid. In a non-expanded state, expandable material 22 readily inserts into ear canal 44. When swollen or expanded, at least portions of the expandable material 22 contact the wall of the ear canal such that motion, such as rotation or twisting, cleans the ear canal wall by transferring the cerumen or contaminants to the expandable material, or loosening the cerumen so that it may be flushed from the ear. Examples of flushing material from the ear may be found in U.S. Patent Application Publication 2010/0179490 to Connely, et al., and U.S. Provisional Application 61/241,978 to the instant inventor, each of which is incorporated herein by reference. It is advantageous if expandable material 22 expands sufficiently in order to contact opposing wall surfaces of ear canal 44, thereby enabling a force to be applied by the expandable material against cerumen or contaminants attached to or adhered to the ear canal wall, thereby facilitating dislodging the cerumen or contaminants, or transfer of same to the expandable material 22.

Although any suitable geometry for the expandable member can be used, a cylindrical geometry is advantageous as it mimics the general shape of the ear canal. The composition of expandable material 22 can be any material that swells when exposed to an irrigation or therapeutic fluid. Examples include polyvinyl acetate, Merocel (a polyvinyl alcetal surgical sponge material which is a registered trademark of Medtronic Xomed, Inc., a Delaware Corporation), cellulose or cellulose based material, collagen, or any other biocompatible material that swells when exposed to the intended therapeutic fluid.

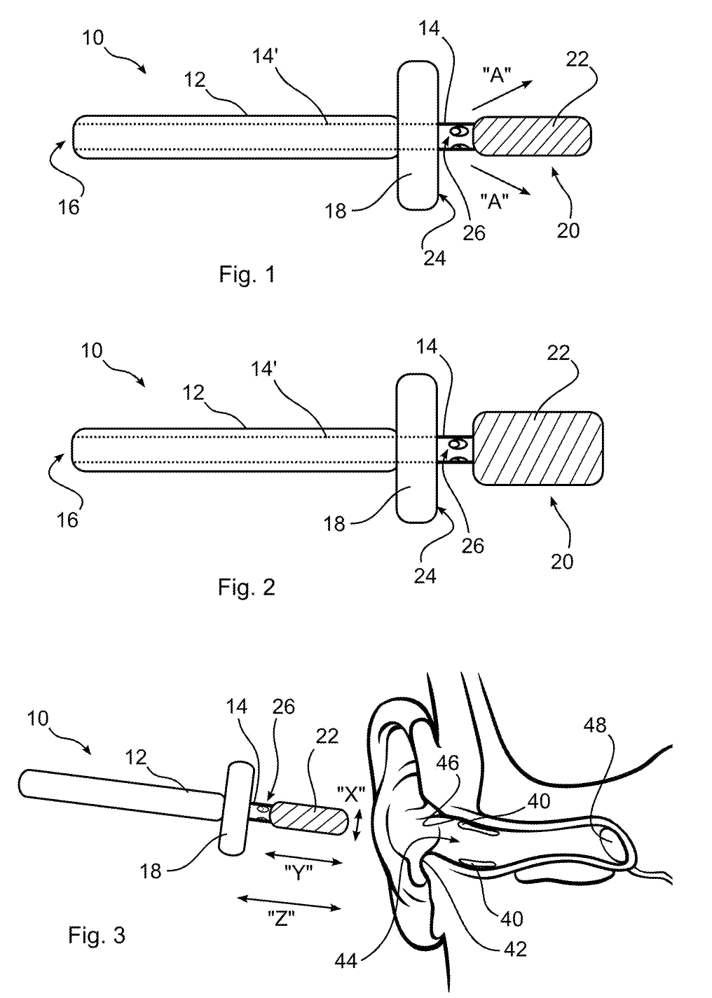

Referring to FIG. 1, a device 10 in accordance with the invention includes a handle 12 adapted to be grasped by the hand of a user. In the illustration, handle 12 has the form of a tube, however any form which is advantageously grasped and manipulated by a hand is contemplated within the spirit and scope of the invention. A fluid reservoir and passage 14′ is disposed within handle 12, operative to contain or conduct fluid. In an embodiment of the invention, an opening 16 is provided at an end of handle 12. A limiter 18 is connected to an end of handle 12, and is operative to contact the tragus 42 of the ear, thereby preventing insertion of device 10 too deeply into the ear canal 44, as explained further below. A support tube 14 extends from limiter 10, on a side opposite handle 12, and is operative to conduct fluid delivered from passage 14′, through limiter 10. An expandable ear canal contacting member 20 is connected to tube 14, and includes an expandable material 22 operative to increase in size when exposed to a fluid. Support tube 14 may extend into handle 12 to form passage 14′, or may be connected to limiter 18 and or tube 12 to establish fluid communication with passage 14′.

In use, device 10 is inserted into the ear canal in need of cleaning, in the orientation shown in FIG. 3. A leading face 24 of limiter 18 contacts the tragus, or cartilaginous structure protecting the opening of the ear canal, and other tissue at the opening of ear canal 44, thereby limiting an extent to which device 10 can be inserted into the ear canal. Once device 10 is inserted into the ear canal, fluid is passed from passage 14′, through or around limiter 18, whereupon the fluid exits through one or more apertures 26, disposed through an outer periphery of tube 14. In an embodiment of the invention, apertures 26 are angled to direct flow against ear canal 44 side walls at an angle, although in accordance with the invention, fluid may also be directed perpendicular to the walls. Apertures 26 are oriented to conduct fluid angularly with respect to an orientation of device 10 within ear canal 44. In this manner, fluid flows against against inner side portions 46 of ear canal 44. Accordingly, fluid flow is not initially directed against ear drum 48, thereby avoiding creating noise and pressure against ear drum 48, increasing safety for the ear, and maintaining comfort for the individual. Apertures 26 are positioned to direct fluid flow under pressure against ear side portions 46, and therefore advantageously have an unobstructed path thereto. Apertures 26 may also be positioned beneath or within expandable material 22, as described further below.

With further reference to FIG. 3, dimensions “X”, “Y”, and “Z” are advantageously selected to fit a particular ear size, for example a child or an adult of the species being treated. Dimension “X” is advantageously selected to be sufficiently small to enable contacting member 20 to be inserted into the ear with a low probability of pushing cerumen further into the ear. The “Z” dimension is selected to ensure device 10 cannot be inserted too far into the ear. Dimension “Y” is selected to be sufficiently long to efficaciously remove excess cerumen. In adult human, the “X”, “Y”, and “Z” dimensions have been determined to advantageously be about ⅛th, ⅜ths, and ⅝ths of an inch (3.2, 9.5, and 15.9 mm), respectively. Limiter 18 has advantageously been determined to be about 9/16ths of an inch (14.3 mm) high. These values correspond, typically, to an inner ear 44 having portions with a height of about ¼ inch (6 mm) in diameter, and an ear canal 44 length of about 1 inch (25.4 mm), for example. These dimensions are merely exemplary, however, and it should be understood that dimensions are selected to fit a user's ear, and to facilitate usage of the device 10 of the invention. The dimensions of contact member 20, for example, may be smaller, while still enabling efficacious use of device 10.

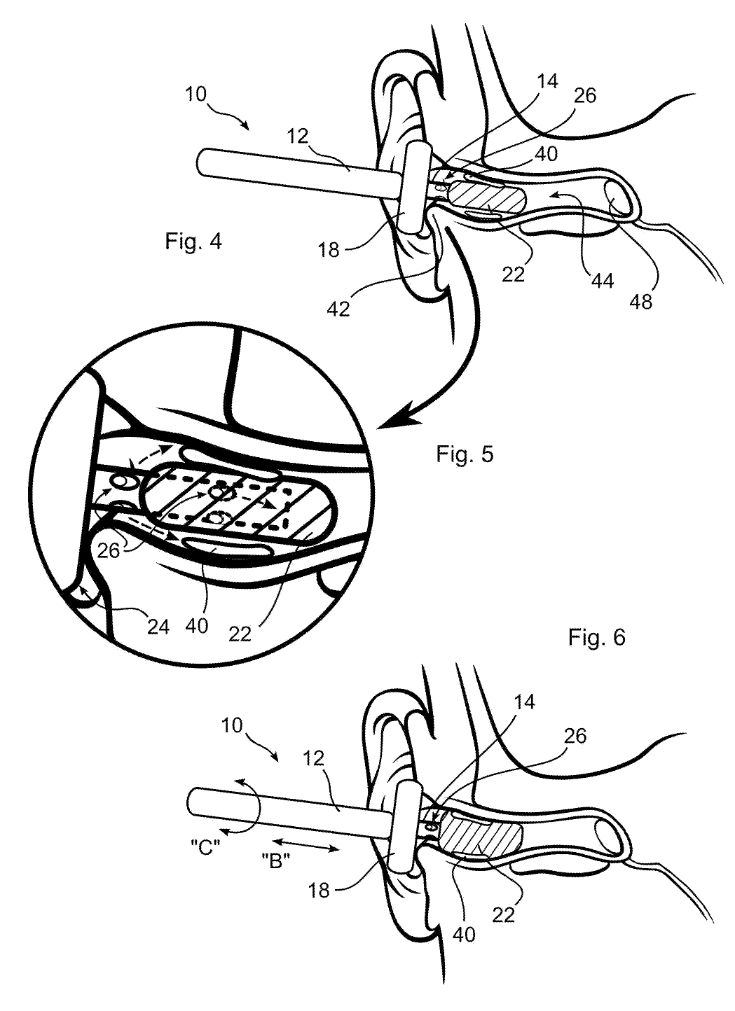

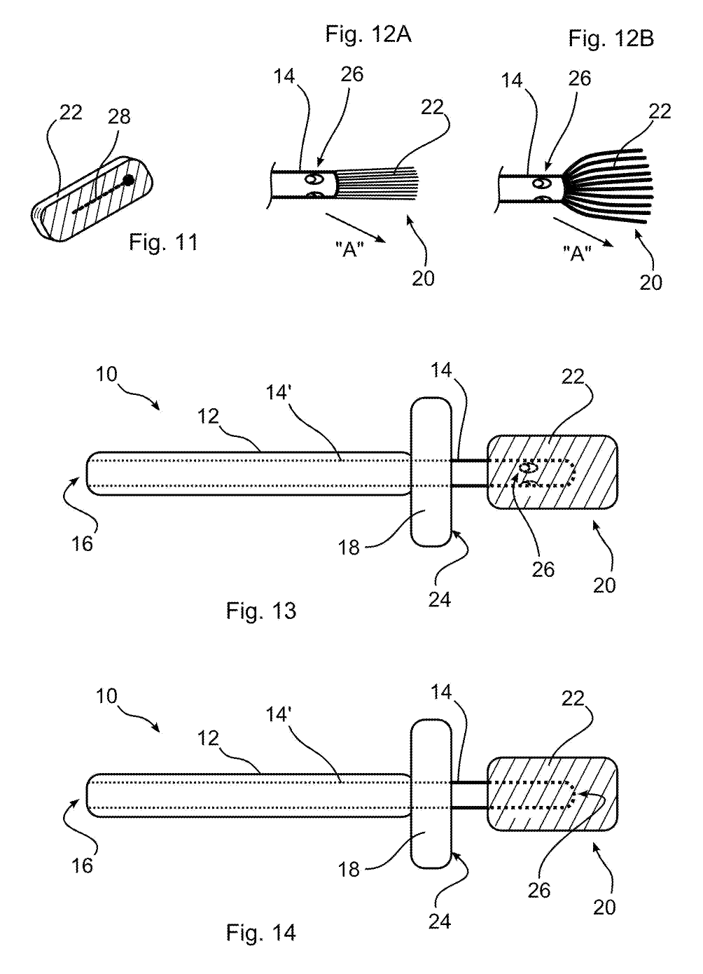

Referring now to FIG. 4, device 10 has been inserted into an ear, with leading face 24 of limiter 18 contacting tragus 42, and contacting member 20, supported on tube 14, disposed within ear canal 44. Expandable material 22 is positioned to overlap at least a portion of cerumen accumulation 40. In an adult human, the distance from tragus 42 to the beginning of ear canal 44, has been found to typically be about ½ inch (12.7 mm), for example. The positioning of contact member 20 with respect to limiter 18 is accordingly selected to locate expandable material 22 within ear canal 44 when limiter 18 contacts tragus 42. Once such positioning has taken place, fluid may be admitted into ear canal 44. As shown in FIG. 5, admitted fluid exits through apertures 26, in the general direction of the dashed arrows, which indicate an advantageously angular trajectory towards a side wall of ear canal 44, and not directly against ear drum 48. It should be understood, however, that fluid may be directed at an angle perpendicular to the walls of ear canal 44. In FIG. 5, additional, optional apertures 26 are illustrated underneath, or within, expandable material 22, provided to hasten moistening of expandable material 22. It should be understood, however, that expandable material 22 may be sufficiently moistened, in some embodiments of the invention, from fluid directed against side walls of ear canal 44. Alternatively, as shown in FIG. 13, apertures 26 may be provided only within expandable material 22, or as shown in FIG. 14, an additional aperture 26 may be provided at the end of support tube 14, either with or without other apertures 26 within expandable material 22, or elsewhere upon support tube 14. In accordance with the foregoing, any combination of aperture 26 locations may be used with any of the various embodiments of the invention.

With reference to FIG. 6, it can be seen that expandable material 22 of contact member 20 has absorbed fluid expelled through apertures 26, and has expanded to contact side wall portions of ear canal 44. In so doing, expandable material contacts cerumen accumulation 40. Accordingly, device 10 may be moved longitudinally with respect to an axis of ear canal 44, as shown by arrow “B”, or may be rotated within ear canal 44, as shown by arrow “C”, to transfer cerumen to contact member 20. The cerumen has been loosened by the action of the fluid expelled through apertures 26, or a mechanical wiping of expandable material 22, or a combination of each, whereby some or all of cerumen 40 transfers to expandable material 22, to be removed as device 10 is withdrawn from the ear. The process of inserting device 10 into ear canal 44, optionally expelling additional fluid through apertures 26, and mechanically wiping cerumen may be repeated until the ear canal is satisfactorily cleaned of cerumen 40 and or other contaminants.

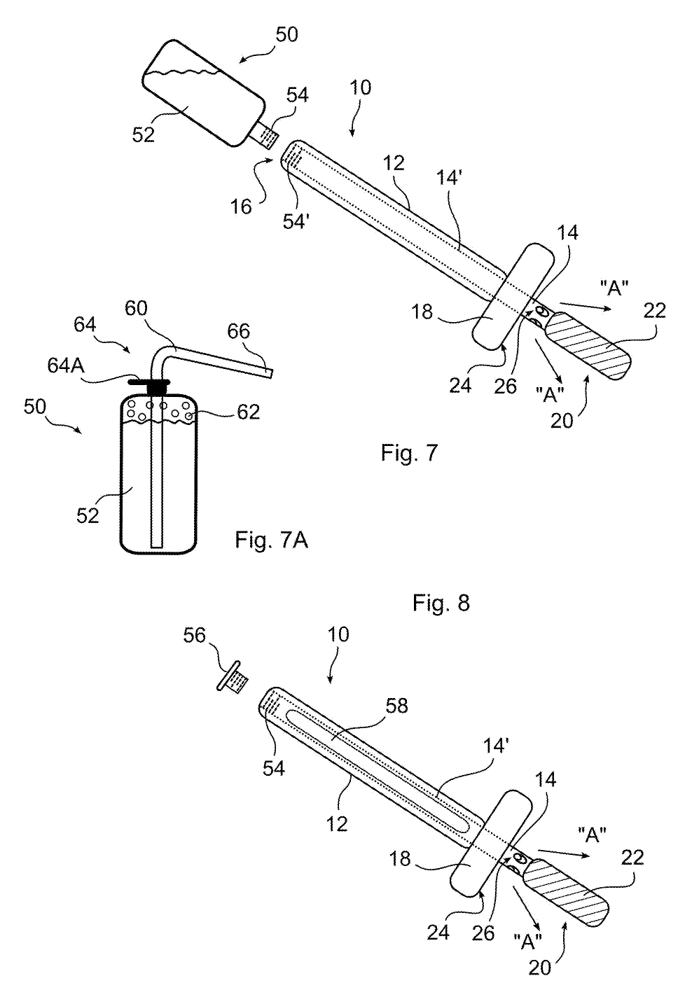

FIG. 7 illustrates one embodiment of the invention, in which fluid is introduced to passage 14′ through opening 16. In this embodiment, a fluid reservoir 50 threadably fastens, as by thread 54, 54′, to passage 14′, although other means of attachment may be used, including a friction fit, or other known method of fluid connection or coupling. Fluid 52 may be transferred to passage 14′, and fluid reservoir 50 removed prior to use, or reservoir 50 may remain connected during use. In the latter circumstance, it may be advantageous if reservoir 50 may be squeezed, in order to provide pressure for fluid flow through apertures 26. If reservoir 50 is to be removed prior to use, a cap, such as threadable cap 56 of FIG. 8 may be used, or other suitable seal. In this event, it may be advantageous if passage 14′ can be squeezed to create pressure. Passage 14′ may also be filled and sealed in any known manner, and a funnel may be used to facilitate filling. If reservoir 50 is provided in the form of a dropper bottle, a mating tapered connection at opening 16 may be advantageous, although any of a variety of shaped openings of sufficient size may be used, as understood by one skilled in the art.

With reference to FIG. 7A, Reservoir 50 may be provided with an internal tube 60, operative to conduct fluid 52 from reservoir 50, whereby pressure within reservoir 50 causes fluid to be driven through internal tube 60 to outside of reservoir 50, for example through opening 16. Pressure may be provided in the form of an internal propellant 62, or by a user squeezing a flexible reservoir 50. Internal tube 60 may optionally be bent or angled to enable maintenance of reservoir 50 in an upright position when connected to opening 16, when device 10 is disposed within the ear. Various connectors 64 may be provided to connect reservoir 50 to device 10, and may include adapters or be adapted to fit industry standard fluid vessels. If internal propellant 62 is used, a trigger 64A is advantageously provided to control fluid flow from reservoir 50. Pressure provided by propellant 62 may be advantageously determined to be appropriate for a particular application, dependent upon fluid viscosity, species, and ear size, for example. Internal tube may be provided with a taper 66 at an outlet end, to facilitate fluid sealing coupling to opening 16, which may be provided with a mating taper.

Alternatively, passage 14′ may filled with a therapeutically beneficial amount of fluid 52 prior to being provided to the user, and passage 14′ may be sealed at a distal and or proximal end of passage 14′, advantageously during manufacturing, and prior to shipment of the device to a user. Passage 14′ is accordingly sized and shaped to contain a suitable amount of fluid 52. Means may be provided to unseal an end of passage 14′ to form a fluid coupling to tube 14, for example a twist lock, pressure sensitive seal, puncturing device, or other method known in the art.

In FIG. 8, an ampule or other fluid chamber 58 may be positioned within passage 14′. In this event, chamber 58 may be squeezed within passage 14′ and thus broken, separated or ruptured, releasing a contained fluid within passage 14′. This is advantageous in the event that materials must be mixed just prior to use, as explained further below. In this embodiment, one or more materials to be mixed are placed within passage 14′, and the remaining elements of a mixture are contained within chamber 58.

In accordance with another embodiment of the invention, a kit may be formed of any or all of the elements of the invention disclosed herein. For example, a kit may be formed with quantities of one or more, optionally in different sizes where applicable, of any of the following: contacting members 20; handle 12; limiter 18; fluid reservoir 50; fluids, optionally of different types; cap 56; propellant 62; connectors 64; cleaning supplies; instructions on paper or audio-visual media; and containers.

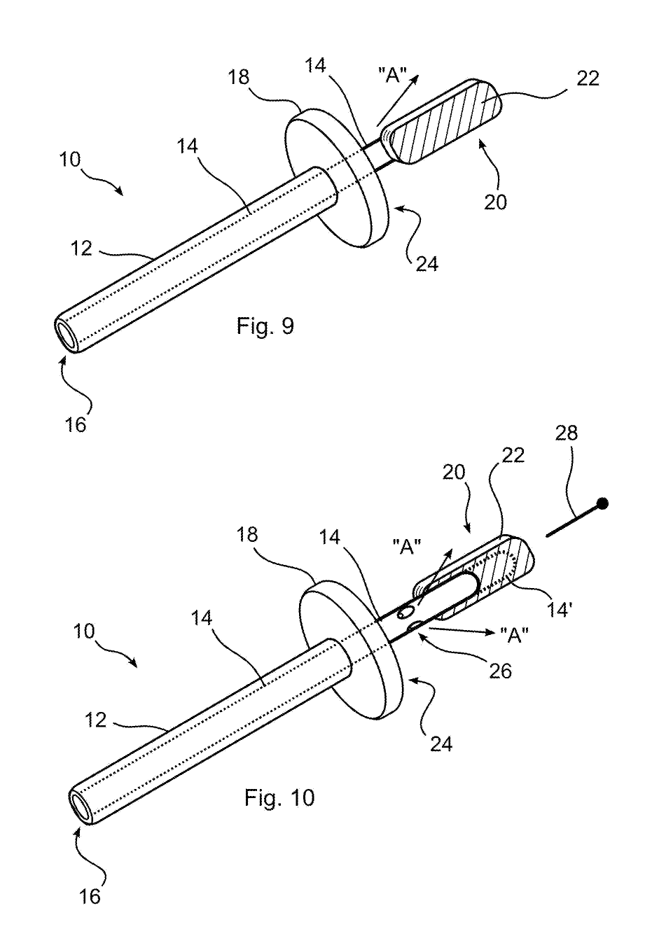

Referring now to FIGS. 9 and 10, a device 10 in accordance with an alternative embodiment of the invention includes contacting member 20, including expandable material 22 formed into a pad connected to support tube 14. Expandable material 22 may be formed into a square or rectangular shape, or any other desired shape. It is advantageous, however, to round or soften any sharp edges, for the comfort and safety of the user. In the embodiment of FIGS. 9 and 10, tube 14 extends into expandable material 22, and may be flattened or otherwise shaped to better insert or fit within expandable material 22, for example, by flattening or shaping an end of tube 14. Alternatively, expandable material 22 may be connected to tube 14 by any known means, including adhesives or fasteners. It is advantageous, however, for there to be no sharp edges or corners which could be injurious to the delicate tissues of the ear and ear canal 44. In one alternative embodiment of the invention, at least a portion of an end of tube 14 is opened, so that fluid may flow into expandable material 22. In this embodiment, apertures 26 may be present, or may be eliminated.

While expandable material 22 is shown in FIGS. 9 and 10 as biased towards a side of tube 14, it should be understood that tube 14 may be contained entirely within expandable material 22, so that no portion of tube 14 is positionable to contact inner ear 44, and or so that as expandable material 22 expands, all sides of inner ear 44 are contacted with even pressure.

As may be seen in FIGS. 10 and 11, an additional support member 28 may be provided, positionable or positioned within expandable material 22, operative to further stiffen expandable material 22, should this be deemed therapeutically advantageous.

With reference to FIGS. 12A and 12B, an alternative form of expandable material 22 is shown, having the form of a brush of bristles. In FIG. 12, expandable material 22 is not moistened or wet, and is accordingly sufficiently narrow to be inserted into ear canal 44 without driving contaminants further into ear canal 44. In FIG. 12B, the bristles of expandable material 22 have been moistened, as by fluid passed through apertures 26, or from contact with fluid applied to inner ear 44 by apertures 26, and have thus expanded to a configuration which improves a contact of expandable material 22 with inner sides of ear canal 44 and contaminants or cerumen 40, accumulated within ear canal 44.

It should be understood that handle 12, limiter 18, and tube 14, and other components of a device in accordance with the invention, may be formed or molded integrally, or may be joined by any known means, including heat, adhesive, ultrasonic energy, or fasteners. Biocompatible materials are advantageously used, particularly in the event that any portion of the device is inadvertently detached and left inside the ear. Biodegradable materials are additionally advantageous for the same reason, and further promote disintegration within landfills. Accordingly, polymeric materials are advantageous, and may be used to form a device which is economically disposable. Alternatively, device 10 may be formed with metal, glass, or other durable material, whereby the device may be cleaned or sterilized by any known means, and contacting member 20, at least, may be advantageously replaced for each patient, connectable to the remainder of the device by screws, fasteners, threads, adhesive, or other means which would be apparent to one skilled in the art.

In one embodiment of the invention, expandable material 22 is a compressed or uncompressed natural or synthetic material, advantageously provided in a sponge structure, operative to expand when contacted by a fluid, for example water, or solutions of alcohol, sodium bicarbonate, one or more antibiotics, one or more antiseptics, or one or more anesthetics. Other therapeutic fluid substances are contemplated within the scope of the invention, as would be understood by one skilled in the art. A wide variety of natural and synthetic materials which are non-injurious to the body, and which are sufficiently soft in both a dry and or compressed form, and in a fluid expanded form, may be advantageously used for expandable material 22, in accordance with the invention. Examples of materials usable for expandable material 22 include cellulosic material, polyvinyl acetate, or Merocel, natural fiber, or synthetic fiber, although many more alternatives would be understood to be usable by one skilled in the art. It is advantageous if the expanded material has an outer surface with a textured or roughened exterior surface, to facilitate removal of cerumen or contaminants attached or adhered to a wall of the ear canal, or to facilitate a transfer of cerumen or contaminants from the ear canal to the expandable material.

The material is advantageously selected for a rate, force and extent of expansion which is comfortable and safe for the ear, the selection of which, beyond the examples suggested herein, is within the abilities of one skilled in the art. In accordance with one embodiment of the invention, an expansion of expandable material 22 from ¼ inch (6.3 mm) to ½ (12.7 mm) inch has been found to be advantageous, or about double in size, although other ratios are contemplated within the invention, particularly where the expanded size may be comfortably compressed by the ear canal to a dimension which conforms thereto. The expandable material 22 may be contained within a sleeve or other container, not shown, until ready for use, particularly if expandable material 22 must be maintained compressed until use.

A variety of substances, in accordance with the invention, may be advantageously used to facilitate removal of cerumen and to clean ear canal 44. A fluid substance is advantageously selected to avoid injury to the ear canal skin and ear drum. In one embodiment, a solution containing 70% isopropyl alcohol is used to soften cerumen, as well as to reduce a potential for infection within the ear, and to promote a reduction of moisture within the ear. In another embodiment, 95% isopropyl alcohol is used, for example in an anhydrous glycerin 5% base. A range of alcohol concentrations may advantageously be used for these purposes, including less than 1% to more than 99% alcohol, depending upon the patient's condition and comfort level, and the therapeutic goals of the practitioner.

A further advantage of an alcohol solution includes use of a device 10 of the invention for drying ear canal 44 after swimming, for example. Moreover, alcohol is antimicrobial, and its use reduces the potential for infection. As understood by practitioners in the art, however, the use of alcohol should be discontinued if there is a burning sensation when used, which may indicate an infection, open wound or sore, or a perforation of ear drum 48. Although there may be temporary discomfort, this is normally transient and not damaging to the ear or hearing.

Alcohol may also be combined with boric acid, for example about 3% boric acid in about 70% alcohol, to treat most bacterial and fungal infections. Of course, the concentrations of boric acid and alcohol may be varied considerably, as would be understood by those skilled in the art. This solution, or any other solution introduced by device 10 into ear canal 44, may be maintained in place, if desired, by placing ear putty at the opening of ear canal 44. Although initially causing discomfort in some individuals, ear putty typically becomes more tolerable over time, and may be maintained for a sufficient period to provide therapeutic benefit, for example, the reduction of an infection.

In one embodiment of the invention, sodium bicarbonate may be placed in fluid chamber 58, whereby breakage or opening of fluid chamber 58 exposes the sodium bicarbonate to a fluid, for example water within, or introduced into, passage 14′, to hydrate and activate the material so that it may be passed into ear canal 44, thereby operative to convey a therapeutic benefit to the ear, including for example a reduction in pH, a reduction of an infection or allergic reaction, or for cleaning. Alternatively, a liquid may be contained within fluid chamber 58, and sodium bicarbonate may be placed within passage 14′. In a similar manner, other chemicals may be combined with liquids and or other chemicals, to produce a fresh composition which offers a therapeutic benefit when introduced into ear canal 44.

In one embodiment of the invention, a temperature gauge or indicator, for example a temperature indicating strip, not shown, may be applied to or incorporated into device 10, in order to indicate whether a fluid to be introduced into the ear is at a comfortable or efficacious temperature, before application.

When sufficient fluid has been introduced into the ear, effluent, advantageously including removed cerumen, will pass out of ear canal 44, and is advantageously collected by a container, such as a kidney basin or similar collector, which may be eliminated if the user is in a suitable environment, for example, a shower. To promote retention of fluid within ear canal 44, a user's head may be tilted slightly backward and towards the nearest shoulder, which further reduces the likelihood of the fluid entering an eye of the user.

Reservoir 50 or tube 14 is squeezed to pass fluid quickly and with sufficient pressure through apertures 26 to loosen cerumen and moisten expandable material 22, but not with such excess pressure that might cause discomfort, or damage ear canal 44 or ear drum 48. The cleaning process is repeated until, if desired, the effluent is free of cerumen or contaminants. Optionally, cleaning may be conducted with sufficient frequency, for example weekly, to maintain ear canal 44 clean, dry, and relatively free of infections, and substantially free of cerumen, although a minor quantity of cerumen may be therapeutically beneficial.

Device 10 of the invention is advantageously used, for example, by individuals; medical practitioners, including ear nose and throat specialists, general practitioners, audiologists, and veterinarians; and athletes, including swimmers.

In yet another embodiment of the invention, apertures 26 may be sized sufficiently small to produce a mist when a liquid is driven therethrough at sufficient pressure. Additionally, a sufficient quantity of apertures 26 may be provided to produce a sufficient quantity of mist over a desired area, for example, to mist the a substantial portion of ear canal 44. A mist promotes an even coating of ear canal 44 with a minimum of fluid. The mist may be maintained within ear canal 44, if desired, by using ear putty, for example.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

All references cited herein are expressly incorporated by reference in their entirety. In addition, unless mention was made above to the contrary, it should be noted that all of the accompanying drawings are not to scale. There are many different features to the present invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention.

What is claimed is:

1. A device for cleaning the ear canal, comprising:
a handle having a distal and a proximal end;
a contact member including a material expandable from a first smaller size to a second larger size upon contact with a fluid, said contact member connected to said proximal end of said handle and sized to be smaller than the ear canal when said expandable material has said first smaller size, whereby said expandable material is insertable into the ear canal;
a limiter connected to said handle, disposed between said handle distal end and said contact member, said limiter including a face having a diameter greater than an opening to the ear canal, whereby said limiter is operative to limit a depth of insertion of said handle and said contact member;
means for passing a fluid through said handle to said contact member when said contact member is disposed within the ear canal, whereby said contact member may be thereby expanded from said first smaller size to said second larger size, whereupon said contact member is sized to contact a wall of the ear canal;
wherein said handle is movable together with said connected contact member to thereby dislodge cerumen or contaminants from attachment to a wall of the ear canal.

2. The device of claim 1, further including a support extension disposed between said limiter and said contact member, operable to support said contact member within the ear canal, and to conduct a fluid.

3. The device of claim 1, further including one or more apertures disposed between said limiter and said contact member, operative to direct fluid to a wall of the ear canal and to said contact member.

4. The device of claim 3, wherein said apertures direct fluid to a wall of the ear canal at an angle less than 90 degrees with respect to an angle of a wall of the ear canal.

5. The device of claim 3, wherein said apertures direct fluid into an interior portion of said expandable material.

6. The device of claim 1, wherein said handle includes a fluid passage operative to contain a fluid and pass said fluid from a distal portion of said handle to a proximal portion of said handle.

7. The device of claim 1, wherein said handle includes a fluid passage operative to contain a fluid and pass said fluid, from a distal portion of said handle to the ear canal, under pressure.

8. The device of claim 1, wherein said second larger size of said contact member has a diameter greater than a diameter of the ear canal.

9. The device of claim 1, wherein said handle and said contact member are not provided with a means for mutual relative disengagement.

10. The device of claim 1, wherein said expandable material is selected from the group consisting of: sponge, cellulosic material, polyvinyl acetate, Merocel, polymer, natural fiber, synthetic fiber.

11. The device of claim 1, wherein said expandable material has a textured or roughened exterior surface.

12. The device of claim 1, further including a reservoir disposable within said handle, said reservoir operable to be ruptured to release a first material into an interior in said handle.

13. The device of claim 12, wherein said interior in said handle is configured to contain a second material, and wherein a combination of said first material and said second material produces a therapeutic substance.

14. The device of claim 6, further including a reservoir connectable to said handle, operative to introduce a material into said fluid passage when said reservoir is connected to said handle.

15. The device of claim 1, wherein said expandable material has the form of a brush.

16. The device of claim 1, wherein said handle and said limiter are not provided with a means for mutual releasable engagement.

17. The device of claim 1, wherein said expandable material is compressed when having said first smaller size.

18. The device of claim 1, wherein said fluid is selected from the group consisting of: alcohol, isopropyl alcohol solution, an isopropyl alcohol and water solution, water, sodium bicarbonate solution, an antibiotic in solution, an antiseptic in solution.

19. A device for cleaning the ear canal, comprising:
a handle having a distal and a proximal end;
a contact member including a material expandable from a first smaller size to a second larger size upon contact with a fluid, said contact member connected to said proximal end of said handle and sized to be smaller than the ear canal when said expandable material has said first smaller size, whereby said expandable material is insertable into the ear canal;
a limiter connected to said handle, disposed between said handle distal end and said contact member, said limiter including a face having a diameter greater than an opening to the ear canal, whereby said limiter is operative to limit a depth of insertion of said handle and said contact member;
a support extension extending between said limiter and said contact member, provided with apertures operative to direct a fluid in a direction of the ear canal when at least a portion of said support extension is disposed within the ear canal, whereby said contact member may expanded, by contact with a fluid, from said first smaller size to said second larger size, whereupon said contact member is sized to contact opposing walls of the ear canal;
wherein said handle is movable together with said connected contact member to thereby dislodge cerumen or contaminants from attachment to a wall of the ear canal.

20. A device for cleaning the ear canal, comprising:
a handle having a distal and a proximal end;
a contact member including a material expandable from a first smaller size to a second larger size upon contact with a fluid, said contact member connected to said proximal end of said handle and sized to be smaller than the ear canal when said expandable material has said first smaller size, whereby said expandable material is insertable into the ear canal;
a limiter connected to said handle, disposed between said handle distal end and said contact member, said limiter including a face having a diameter greater than an opening to the ear canal, whereby said limiter is operative to limit a depth of insertion of said handle and said contact member;
a support extension extending between said limiter and said contact member, provided with at least one aperture operative to direct a fluid in a direction of the ear canal when at least a portion of said support extension is disposed within the ear canal, whereby said contact member may expanded, by contact with a fluid, from said first smaller size to said second larger size, whereupon said contact member is sized to contact opposing walls of the ear canal;
a reservoir connectable with said handle, operative to contain a fluid and pass the fluid to said at least one aperture;
wherein said handle is movable together with said connected contact member to thereby dislodge cerumen or contaminants from attachment to a wall of the ear canal.
